(19) United States
(12) Patent Application Publication
(10) Pub. No.: US 2025/0161107 A1
(43) Pub. Date: May 22, 2025

(54) Reusable Thermal Contact Pad

(71) Applicant: C. R. Bard, Inc., Franklin Lakes, NJ (US)

(72) Inventors: Madeline Stich, Thornton, CO (US); Michael R. Hoglund, Windsor, CO (US); Gabriel A. Johnston, Broomfield, CO (US); Sean E. Walker, Platteville, CO (US); Rebecca D. Salisbury, Lafayette, CO (US)

(21) Appl. No.: 18/832,782

(22) PCT Filed: Jan. 24, 2022
(86) PCT No.: PCT/US2022/013569

§ 371 Date: Jul. 24, 2024

(51) Int. Cl. A61F 7/02; A61F 7/00

(52) U.S. Cl. CPC A61F 7/02; A61F 7/0085; A61F 2007/0018; A61F 2007/0041; A61F 2007/0054; A61F 2007/0234

(57) ABSTRACT

Disclosed herein are medical systems, pads, and methods for exchanging thermal energy between a circulating fluid and a patient. Some pads are configured multiple uses on the same patient or across multiple different patients. Some pads include multiple layers of hydrogel disposed on one or both sides a fluid compartment. Removable hydrogel layers expose other hydrogel layers for subsequent uses of the pad. Removable storage liners encapsulate one or multiple hydrogel layers. Separation liners between adjacent hydrogel layers isolate the adjacent hydrogels from each other and removal of the separation liner extends the operational life a hydrogel layer. Adhesive patches secure the pads to the patient including adhesive patches extending through openings of a pad. Some pads are configured for disinfection, sterilization and/or laundering. In an embodiment, the fluid is air and a vacuum within a compartment of the pad secures the pad to the patient.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems include a TTM fluid control module coupled to at least one contact pad via a fluid deliver line. One such system including a thermal contact pad is disclosed in U.S. Publication No. 2020/0155341, titled “Medical Pad and system for Thermotherapy,” filed Oct. 9, 2019, which is incorporated herein by reference in its entirety.

A thermal contact pad is typically configured for use over a small number of applications such as a single use or application. In some instances, thermal contact pads may be come soiled and/or contaminated from use. However, replacing thermal contact pads after each use can become costly for the healthcare provider. Disclosed here are systems, thermal contact pads, and methods for utilizing a thermal contact pad over an extended period of time, including multiple TTM therapies across multiple patients.

### SUMMARY OF THE INVENTION

Briefly summarized, disclosed herein is a patient contact system for exchanging thermal energy between a targeted temperature management (TTM) fluid and a patient. According to some embodiments, the patient contact system includes a gown assembly, where the gown assembly includes a gown configured for placement on the patient and a thermal contact pad coupled with the gown. The pad includes a fluid compartment configured to receive a TTM fluid from a TTM system module via a fluid delivery line, where the TTM system module is configured to circulate the TTM fluid within the fluid compartment during the performance of a TTM therapy. The patient contact system further includes a hydrogel applied to a patient contact side of the pad. The pad is disposed on an inside surface of the gown to facilitate thermal contact of the fluid compartment of the pad with the patient.

In some embodiments, the patient contact system is configured for multiple uses on a single patient and/or multiple uses across different patients.

The gown assembly may be configured for disinfection and/or sterilization and the sterilization may include autoclaving. The gown assembly may also be configured for laundering within an automated laundering machine.

The hydrogel applied to the pad may be a first portion of hydrogel dispensed from a separate container.

In some embodiments, the gown assembly includes a plurality of thermal contact pads. The plurality of thermal contact pads may include a first pad coupled with the gown so as to be located adjacent a first side of a torso of the patient when the gown assembly is placed on the patient and a second pad coupled with the gown so as to be located adjacent a second side of the torso opposite the first side when the gown assembly is placed on the patient. The plurality of thermal contact pads may further include a third pad coupled with the gown so as to be located adjacent a first thigh of the patient when the gown assembly is placed on the patient and a fourth pad coupled with the gown so as to be located adjacent a second thigh of the patient when the gown assembly is placed on the patient. In some embodiments, one or more of the plurality of thermal contact pads may be removably coupled with the gown assembly.

In some embodiments, the plurality of thermal contact pads are fluidly interconnected. In some embodiments, two or more of the plurality of thermal contact pads are individually coupled with the TTM system module. In some embodiments, one or more of the plurality of thermal contact pads are fluidly disconnectable from the remaining pads.

In some embodiments, the gown assembly includes a plurality of fastening devices, where one or more of the plurality of fastening devices are configured to secure the gown to the patient. One or more of the plurality of fastening devices may also be configured to secure one or more of the plurality of pads in thermal contact with the patient.

In some embodiments, a support structure is disposed within the fluid compartment to define a minimum thickness of the fluid compartment.

Also disclosed herein is a method of providing one or more targeted temperature management (TTM) therapies to one or more patients. The method includes applying a gown assembly to a first patient, where the gown assembly includes a gown configured for placement on a patient and a plurality of thermal contact pads coupled with the gown. Each pad includes a fluid compartment configured to receive a TTM fluid from a TTM system module via a fluid delivery line, where the TTM system module is configured to circulate the TTM fluid within the fluid compartment during the performance of the TTM therapy. The method further includes: (i) dispensing a hydrogel from a hydrogel container onto a patient contact side of each pad, (ii) securing each pad in thermal contact with the first patient so that the hydrogel is disposed between each corresponding fluid compartment and the first patient, (iii) coupling the gown assembly to the TTM system module, and (iv) circulating the TTM fluid through the fluid compartment of each pad to perform a first TTM therapy.

In some embodiments, the method further includes: (i) removing the gown assembly from the first patient, (ii) applying the gown assembly to a second patient, (iii) dispensing the hydrogel from the hydrogel container onto each pad a second time, (iv) securing each pad in thermal contact with the second patient, and (v) circulating the TTM fluid through the fluid compartment of each pad a second time. In some embodiments of the method, the first patient and the second patient are different patients.

In some embodiments, the method further includes disinfecting the gown assembly between the first TTM therapy and the second TTM therapy. In some embodiments, the method further includes sterilizing the gown assembly between the first TTM therapy and the second TTM therapy. In some embodiments, the method further includes laundering the gown assembly with an automated laundering machine between the first TTM therapy and the second TTM therapy.

In some embodiments, the method further includes decoupling a thermal contact pad from the gown assembly, and coupling a replacement thermal contact pad with the gown assembly.

Also disclosed herein is a patient contact system the includes a thermal contact pad configured for placement on a patient, where the pad includes a fluid compartment configured to receive a TTM fluid from a TTM system module via a fluid delivery line, the TTM system module configured to circulate the TTM fluid within the fluid compartment during the performance of the TTM therapy. The pad further includes a plurality openings extending between a topside and an underside of the pad. The patient contact system further includes a plurality of first adhesive patches, where each first adhesive patch is disposed over a top of each opening. In use, a portion of the first adhesive patch is depressed through the corresponding opening so as to adhere the portion of the first adhesive patch to the patient to secure the pad to the patient. Each adhesive patch is configured for separation from the patient and subsequent re-adherence to the patient.

In some embodiments, the system is configured for multiple uses on a single patient including and in further embodiments, the system is configured for multiple uses across different patients. In some embodiments, the pad is configured for disinfection and or sterilization and the sterilization may include autoclaving.

In some embodiments, the system further includes a plurality of second adhesive patches, where each second adhesive patch includes a first portion coupled with the pad on the topside of the pad along a perimeter edge of the pad and a second portion extending away from the perimeter edge, where, in use, the second portion is adhered to the patient to secure the pad to the patient.

In some embodiments of the system, a hydrogel is applied to the pad across an underside of the pad, and in some embodiments, a hydrogel liner disposed between the hydrogel and the fluid compartment. In some embodiments, the hydrogel liner is removably attached to the fluid compartment. The hydrogel liner may include a plurality of liner openings shaped and positioned to correspond to the openings of the pad.

In some embodiments of the system, the hydrogel is sealably encapsulated within a hydrogel compartment, where the compartment is defined by the hydrogel liner disposed across a topside the hydrogel and a storage liner disposed across an underside of the hydrogel, and in use, the storage liner is removed from the pad to expose the hydrogel.

In some embodiments of the system, the fluid compartment includes a support structure disposed therein to define a minimum thickness of the fluid compartment.

Also disclosed herein is a method of providing one or more targeted temperature management (TTM) therapies to one or more patients, including: (i) applying a thermal contact pad to a first patient, the thermal contact pad including a plurality openings extending between a topside and an underside of the pad; (ii) applying a plurality of first adhesive patches, each first adhesive patch disposed over a top of each opening; (iii) depressing a portion of each first adhesive patch through the corresponding opening to adhere to the first adhesive patch to the first patient thereby securing the pad to the first patient; (iv) coupling a fluid compartment of the thermal contact pad to a TTM system module, the fluid compartment configured to receive a TTM fluid from a TTM system module via a fluid delivery line, the TTM system module configured to circulate the TTM fluid within the fluid compartment during the performance of the TTM therapy; and (v) circulating the TTM fluid through the fluid compartment of the pad to perform a first TTM therapy.

In some embodiments, the method includes applying a first volume of hydrogel to the pad across an underside of the pad. In further embodiments of the method, the pad includes a first hydrogel liner coupled with the fluid compartment, and the first volume of hydrogel is applied to the first hydrogel liner. In some embodiments of the method, the first volume of hydrogel is sealably encapsulated within a first hydrogel compartment that is defined by the first hydrogel liner and a first storage liner disposed across an underside of the first volume of hydrogel, and the method further includes removing the first storage liner from the pad to expose the first volume of hydrogel.

In some embodiments, the method further includes: (i) removing the pad from the first patient, (ii) removing the first adhesive patches from the pad, (iii) applying the pad to a second patient, (iv) applying a second plurality of first adhesive patches to the pad, (v) depressing a portion of each first adhesive patch of the second plurality through the corresponding opening to adhere to the first adhesive patches of the second plurality to the first patient thereby securing the pad to the second patient, and (vi) circulating the TTM fluid through the fluid compartment of the pad a second time to perform a second TTM therapy. In some embodiments of the method, the first patient and the second patient are different patients.

In some embodiments, the method further includes disinfecting the pad between the first TTM therapy and the second TTM therapy. In some embodiments, the method further includes sterilizing the pad between the first TTM therapy and the second TTM therapy.

In some embodiments, the method further includes removing the first volume of hydrogel from the pad after the first TTM therapy and applying a second volume of hydrogel to the pad prior to the second TTM therapy.

In some embodiments, the method further includes removing the first hydrogel liner from the pad after the first TTM therapy, attaching a second hydrogel compartment to the pad, and removing a second storage liner from the second hydrogel compartment to expose the second volume of hydrogel.

Also disclosed herein is a medical pad, including: (i) a fluid compartment configured to receive a TTM fluid from a TTM system module via a fluid delivery line, the TTM system module configured to circulate the TTM fluid within the fluid compartment during the performance of a TTM therapy; (ii) a first hydrogel layer coupled with the fluid compartment on an underside of the fluid compartment; and (iii) a second hydrogel layer coupled with the first hydrogel layer on an underside of the first hydrogel layer.

In some embodiments of the pad, the second hydrogel layer is removably coupled with the first hydrogel layer. In some embodiments, a separation liner is disposed between the first hydrogel layer and the second hydrogel layer, and in further embodiments, the separation liner is coupled with the fluid compartment to sealably encapsulate the first hydrogel layer.

In some embodiments of the pad, the separation liner includes a pull tab extending away from a perimeter edge of the separation liner to facilitate removal of the separation liner together with the second hydrogel layer.

In some embodiments, the pad further includes a first liner disposed between the fluid compartment and the first hydrogel layer and in further embodiments, the separation liner is coupled with the first liner to sealably encapsulate the first hydrogel layer.

In some embodiments, the pad further includes a storage liner disposed across an underside of the second hydrogel layer, where the storage liner coupled with one or more of the fluid compartment, the separation liner, or the first liner to sealably encapsulate the second hydrogel layer. In some embodiments, the pad further includes a foam layer coupled with the fluid compartment on a topside of the fluid compartment.

Also disclosed herein is a method of providing one or more targeted temperature management (TTM) therapies to one or more patients. The method includes providing a thermal contact pad that includes: (i) a fluid compartment configured to receiver a TTM fluid from the TTM system module via a fluid delivery line, where the TTM system module is configured to circulate the TTM fluid within the fluid compartment during the performance of a TTM therapy; (ii) a first hydrogel layer coupled with the fluid compartment; and (iii) a second hydrogel layer coupled with first hydrogel layer opposite the fluid compartment. The method further includes applying the pad to a first patient so that the second hydrogel layer is disposed in contact with the first patient, coupling the fluid compartment to the TTM system module, and circulating the TTM fluid through the fluid compartment to perform a first TTM therapy.

In some embodiments, the method further includes: (i) removing the pad from the first patient; (ii) removing the second hydrogel layer from the pad, thereby exposing the first hydrogel layer; (iii) applying the pad to a second patient so that the first hydrogel layer is disposed in contact with the second patient; and (iv) circulating the TTM fluid within the fluid compartment a second time to perform a second TTM therapy. In some embodiments of the method, the first patient and the second patient are different patients.

In some embodiments of the method, the pad further includes a separation liner disposed between the first hydrogel layer and the second hydrogel layer, and the method further includes removing the separation liner from the pad together with the second hydrogel layer.

In some embodiments of the method, the separation liner is coupled with the fluid compartment to sealably encapsulate the first hydrogel layer.

In some embodiments of the method, the pad further includes a storage liner disposed across an underside of the second hydrogel layer, and the method further includes removing the storage liner from the pad to expose the second hydrogel layer prior to applying the pad to the first patient. In some embodiments of the method, the storage liner is coupled with one or more of the fluid compartment or the separation liner to sealably encapsulate the second hydrogel layer.

Also disclosed herein is a medical pad, including a foam layer and a first fluid compartment coupled with the foam layer on a first side of the foam layer, where the first fluid compartment is configured to receive a TTM fluid from a TTM system module via a first fluid delivery line, and where the TTM system module is configured to circulate the TTM fluid within the first fluid compartment during the performance of a first TTM therapy. The pad further includes: (i) a first hydrogel layer coupled with the first fluid compartment opposite the foam layer; (ii) a second fluid compartment coupled with the foam layer on a second side of the foam layer opposite the first side, where the second fluid compartment is configured to receive the TTM fluid from the TTM system module via a second fluid delivery line; and (iii) a second hydrogel layer coupled with the second fluid compartment opposite the foam layer.

In some embodiments of the pad, the first hydrogel layer is removably coupled with the first fluid compartment and in some embodiments, the second hydrogel layer is removably coupled with the second fluid compartment.

In some embodiments, the pad further includes a first liner disposed between the first hydrogel layer and the first fluid compartment, and in further embodiments, the first liner is removably attached to the first fluid compartment.

In some embodiments, the pad further includes a second liner disposed between the second hydrogel layer and the second fluid compartment, and in further embodiments, the second liner is removably attached to the second fluid compartment.

In some embodiments of the pad, the first liner includes a first pull tab extending away from a perimeter edge of the first liner to facilitate removal of the first liner from the first fluid compartment, and similarly in further embodiments, the second liner includes a second pull tab extending away from a perimeter edge of the second liner to facilitate removal of the second liner from the second fluid compartment.

In some embodiments, the pad further includes a first storage liner disposed across an outerside of the first hydrogel layer, where the first storage liner is coupled with one or more of the first fluid compartment, or the first liner to sealably encapsulate the first hydrogel layer. Similarly, in some embodiments, the pad further includes a second storage liner disposed across an outerside of the second hydrogel layer, where the second storage liner is coupled with one or more of the second fluid compartment, or the second liner to sealably encapsulate the second hydrogel layer.

Also disclosed herein is a method of providing one or more targeted temperature management (TTM) therapies to one or more patients, that includes providing a thermal contact pad, where the pad includes: (i) a first fluid compartment configured to receive a TTM fluid from a TTM system module via a first fluid delivery line, where the TTM system module is configured to circulate the TTM fluid within the first fluid compartment during the performance of a first TTM therapy; (ii) a first hydrogel layer coupled with the first fluid compartment; (iii) a second fluid compartment coupled with the first fluid compartment opposite the first hydrogel layer, the second fluid compartment configured to receive the TTM fluid from the TTM system module via a second fluid delivery line during the performance of a second TTM therapy; and (iv) a second hydrogel layer coupled with the second fluid compartment opposite the first fluid compartment. The method further includes applying the pad to a first patient so that the first hydrogel layer is disposed in contact with the first patient, coupling the first fluid compartment to the TTM system module, and circulating the TTM fluid through the first fluid compartment to perform the first TTM therapy.

In some embodiments of the method, the pad further includes a first storage liner disposed across an outerside of the first hydrogel layer, and the method further includes removing the first storage liner from the pad to expose the first hydrogel layer prior to applying the pad to the first patient.

In some embodiments, the method further includes: (i) removing the pad from the first patient; (ii) inverting the pad; (iii) applying the pad to a second patient so that the second hydrogel layer is disposed in contact with the second patient; and (iv) circulating the TTM fluid through the second fluid compartment to perform the second TTM therapy. In some embodiments, the first patient and the second patient are different patients.

In some embodiments of the method, the first hydrogel layer is removably coupled with the first fluid compartment, and the method further includes removing the first hydrogel layer from the pad after removing the pad from the first patient.

In some embodiments of the method, the pad further includes a first liner disposed between the first hydrogel layer and the first fluid compartment, and the method further includes removing the first liner from the pad together with the first hydrogel layer.

In some embodiments of the method, the pad further includes a second storage liner disposed across an outerside of the second hydrogel layer, and the method further includes removing the second storage liner from the pad to expose the second hydrogel layer prior to applying the pad to the second patient.

Also disclosed herein is a medical pad for exchanging thermal energy between a targeted temperature management (TTM) fluid and a patient, where the pad includes: (i) a fluid compartment configured to receiving a TTM fluid from a TTM system module via a fluid delivery line, where the TTM system module is configured to circulate the TTM fluid within the fluid compartment during the performance of a TTM therapy; (ii) a first hydrogel layer coupled with the fluid compartment on an underside of the fluid compartment; (iii) a second hydrogel layer coupled with the first hydrogel layer on an underside of the first hydrogel layer; and (iv) a separation liner disposed between the first hydrogel layer and the second hydrogel layer, where the separation liner isolates the second hydrogel layer from the first hydrogel layer and where the separation liner is removably coupled with the first and second hydrogel layers.

In some embodiments of the pad, removing the separation liner from between to first and second hydrogel layers provides for the migration of one or more constituents of the first hydrogel layer into the second hydrogel layer. In some embodiments, the migration of the one or more constituents extends an operational life of the pad. In further embodiments, the migration of the one or more constituents provides for extending a duration of the TTM therapy. In some embodiments, the migration of the one or more constituents facilitates a rehydration of the second hydrogel layer. In still other embodiments, a chemical composition of the second hydrogel layer is different from a chemical composition of the first hydrogel layer.

In some embodiments of the pad, the separation liner is coupled with the fluid compartment to sealably encapsulate the first hydrogel layer. In some embodiments, the separation liner includes a pull tab extending away from a perimeter edge of the separation liner to facilitate removal of the separation liner together with the second hydrogel layer.

In some embodiments, the pad further includes a liner disposed between the fluid compartment and the first hydrogel layer. In further embodiments, pad further includes a storage liner disposed across an underside of the second hydrogel layer, where the storage liner is coupled with one or more of the fluid compartment, the separation liner, or the liner to sealably encapsulate the second hydrogel layer.

In some embodiments, the pad further includes a foam layer coupled with the fluid compartment on a topside of the fluid compartment opposite the first hydrogel layer.

Also disclosed herein is a method of providing targeted temperature management (TTM) therapy to a patient, which includes providing a thermal contact pad. The pad includes: (i) a fluid compartment configured to receive a TTM fluid from a TTM system module via a fluid delivery line, where the TTM system module is configured to circulate the TTM fluid within fluid compartment during the performance of a TTM therapy; (ii) a first hydrogel layer coupled with the fluid compartment; (iii) a separation liner coupled with the first hydrogel layer opposite the fluid compartment; and (iv) a second hydrogel layer coupled with the separation liner opposite the first hydrogel layer, where the separation liner isolates the second hydrogel layer from the first hydrogel layer, and the separation liner is removably coupled with the first and second hydrogel layers. The method further includes applying the pad to the patient so that the second hydrogel layer is disposed in contact with the patient, coupling the fluid compartment to the TTM system module, and circulating the TTM fluid within the fluid compartment to perform the TTM therapy.

In some embodiments, the method further includes removing the separation liner from between the first and second hydrogel layers to allow one or more constituents of the first hydrogel layer to migrate into the second hydrogel layer.

In some embodiments of the method, the pad the further includes a storage liner disposed across an underside of the second hydrogel layer, and the method further includes removing the storage liner to expose the second hydrogel layer.

Also disclosed herein is a medical system for providing a targeted temperature management (TTM) therapy to a patient. According to some embodiments, the medical system includes a targeted temperature management (TTM) system module configured to provide a flow of TTM air at a defined pressure and temperature in accordance with a TTM therapy and a thermal contact pad fluidly coupled with the system module, where the pad is configured for placement on a patient. The pad is configured to sealably couple with the patient along a circumference of the pad to form a compartment between a top wall of the pad and a skin surface of the patient, where the compartment is configured to receive the TTM air from the system module via a bidirectional air delivery line for circulation of the TTM air within the compartment, and where the defined pressure is a vacuum causing a downward force on the pad to secure the pad to the patient during the TTM therapy.

In some embodiments of the system, the pad includes a support structure extending downward from the top wall within the compartment, and the support structure defines a minimum distance between the skin surface and the top wall.

In some embodiments of the system, the support structure defines a plurality channels for flow of the TTM air during circulation through the compartment between an inlet port and an outlet port.

In some embodiments, the system includes a plurality of the thermal contact pads coupled with the system module, and in some embodiments, each pad of the plurality of the pads is individually coupled with the system module.

Also disclosed herein is a method of providing targeted temperature management (TTM) therapy to a patient, the includes: (i) applying a thermal contact pad to the patient, where the pad forms a circumferential seal with a skin surface of the patient to define a compartment between a top wall of the pad and the skin surface; (ii) fluidly coupling the pad with a TTM system module via a bidirectional air delivery line, where the TTM system module is configured to circulate TTM air within the compartment; (iii) defining a vacuum within the fluid compartment to secure the pad to the patient; and (iv) circulating the TTM air within the compartment at the defined temperature to perform the TTM therapy.

In some embodiments, the method further includes removing the vacuum from the compartment to release the pad from the patient.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management (TTM) system including a plurality of thermal contact pads coupled with a patient, in accordance with some embodiments;

FIG. 2A illustrates a first embodiment of a patient contact system including a front view of a gown assembly incorporating a plurality of thermal contact pads that may be employed with the system of FIG. 1, in accordance with some embodiments;

FIG. 2B illustrates an inside view of the view of the gown assembly of FIG. 2A, in accordance with some embodiments;

FIG. 2C illustrates a cross-sectional view of the thermal contact pad of the gown assembly of FIG. 2A cut along sectioning lines 2C-2C, in accordance with some embodiments;

FIG. 3A illustrates a second embodiment of a patient contact system that may be employed with the system of FIG. 1 including a thermal contact pad having openings extending through the pad, in accordance with some embodiments;

FIG. 3B illustrates a cross-sectional view of the thermal contact pad of FIG. 3A cut along sectioning lines 3B-3B, in accordance with some embodiments;

FIG. 3C illustrates a top view of a hydrogel container of FIG. 3A, in accordance with some embodiments;

FIG. 3D illustrates a cross-sectional view of the hydrogel container of FIG. 3C cut along sectioning lines 3D-3D, in accordance with some embodiments;

FIG. 4A illustrates another embodiment of a thermal contact pad that may be employed with the system of FIG. 1, in accordance with some embodiments;

FIG. 4B illustrates a cross-sectional view of the pad of FIG. 4A cut along sectioning lines 4B-4B, in accordance with some embodiments;

FIG. 5A illustrates another embodiment of a thermal contact pad that may be employed with the system of FIG. 1, in accordance with some embodiments;

FIG. 5B illustrates a cross-sectional view of the pad of FIG. 5A cut along sectioning lines 5B-5B, in accordance with some embodiments;

FIG. 6A illustrates another embodiment of a thermal contact pad that may be employed with the system of FIG. 1, in accordance with some embodiments;

FIG. 6B illustrates a cross-sectional view of the pad of FIG. 6A cut along sectioning lines 6B-6B, in accordance with some embodiments;

FIG. 7A illustrates another embodiment of a TTM system, in accordance with some embodiments;

FIG. 7B illustrates a top view of a thermal contact pad of the TTM system of FIG. 7A, in accordance with some embodiments; and

FIG. 7C illustrates a cross-sectional view of the pad of FIG. 7B cut along sectioning lines 7C-7C, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled with” refer to any form of interaction between two or more entities, including mechanical, fluid, and thermal interaction. Two components may be connected to or coupled with each other even though they are not in direct contact with each other. For example, two components may be coupled with each other through one or more intermediate components.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

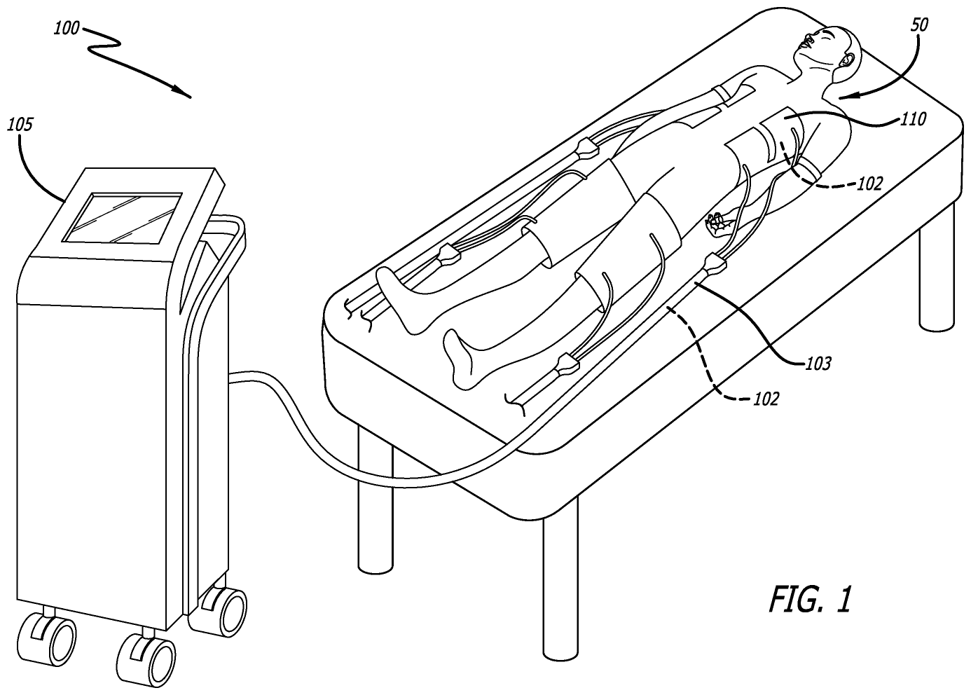

FIG. 1 illustrates a targeted temperature management (TTM) system 100 connected to a patient 50 for administering TTM therapy to the patient 50 which may include a cooling and/or warming of the patient 50, in accordance with some embodiments. The TTM system 100 includes a TTM module 105 for preparing and delivering a TTM fluid 102 to a thermal contact system 110 which may, in some embodiments, include one more thermal contact pads. The TTM system 100 includes a bidirectional fluid deliver line (FDL) 103 extending from the TTM module 105 to thermal contact system 110 to provide for flow of the TTM fluid 102 between the TTM module 105 and the contact system 110.

In use, the TTM module 105 prepares the TTM fluid 102 for delivery to the contact system 110 by heating or cooling the TTM fluid 102 to a defined temperature in accordance with a prescribed TTM therapy. The TTM module 105 circulates the TTM fluid 102 within/through the contact system 110 to facilitate thermal energy exchange with the patient 50. During the TTM therapy, the TTM module 105 may continually control the temperature of the TTM fluid 102 toward a target TTM temperature. As shown, the contact system 110 may be applied to different body parts of the patient 50. As such, the contact system 110 may be available in different configurations, such as sizes and shapes, for example, to accommodate the different body parts.

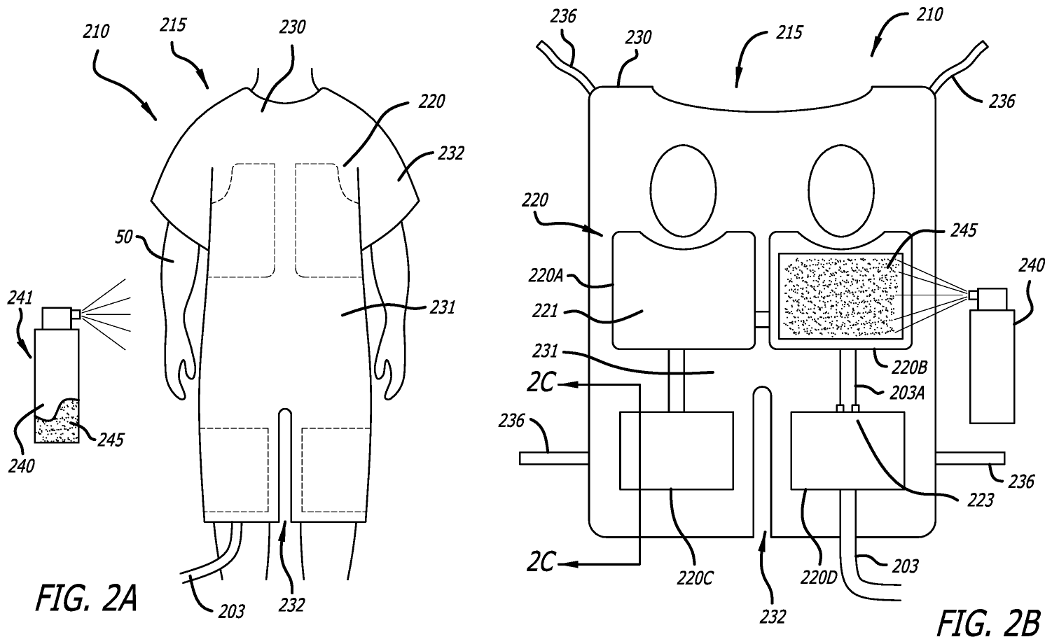

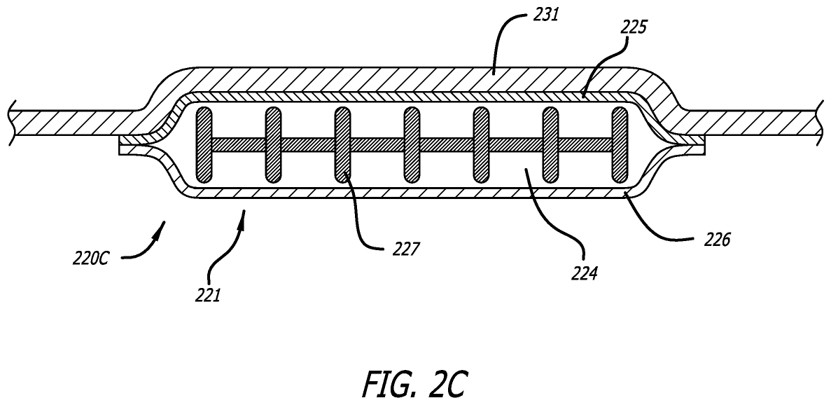

FIGS. 2A-2C illustrate a first embodiment of a thermal contact system 210 that generally includes a gown assembly and a hydrogel source. FIG. 2A is a front view of the gown assembly 215 together with the hydrogel source 240. The gown assembly 215 includes a gown 230 and one or more thermal contact pads (pads) 220 coupled with gown 230. The thermal contact system 210 includes an FDL 203 for fluidly coupling the pads 220 with the system module 105 (see FIG. 1). The hydrogel source 240 includes a container 241 having hydrogel 245 disposed therein.

The gown 230 is configured for placement on a patient 50. In some embodiments, the gown 230 may include a main fabric panel (panel) 231 having sleeves 232 coupled therewith for receiving the patient's arms therethrough. In the illustrated embodiment, the gown 230 (or more specifically the panel 231) is configured for placement across a front side of the patient 50 and wrapping around to a back side of the patient 50. In an alternative embodiment, the gown 230 may be configured for placement across the back side of the patient 50 and wrapping around to the front side of the patient 50. The pads 220 may be coupled with the panel 231 so that an underside of the pads 220 may be placed in direct contract with a skin of the patient 50. In some embodiments, the pads 220 may be disposed on an underside of the panel 231. In some embodiments, the panel 231 may include a slit 232 extending upward from a bottom edge of the panel 231 to accommodate wrapping portions of panel 231 around (or partially around) one or both legs of the patient 50.

The thermal contact system 210 may be configured for multiple uses with a single patient or multiple uses across multiple patients. The gown assembly 215 may be configured for repeated cleaning, including disinfection and/or sterilization. In some embodiments, the gown assembly 215 may be configured for repeated laundering via an automated laundering machine. In some embodiments, the gown assembly 215 may be configured for repeated autoclaving.

FIG. 2B is an underside view of the gown assembly 215. The pads 220 are shown coupled with the panel 231 on the underside thereof so that the pads 220 may contact the skin directly when the gown assembly 215 is placed on the patient 50. In some embodiments, the gown 230 may be configured for securing one portion of the gown 230 with another portion. As such, the gown 230 may include fastening devices 236. The fastening devices 236 may include straps, ties, buttons, snaps, hook and loop patches, or any other suitable fastening devices. In some embodiments, the fastening devices 236 may facilitate exerting a downward force on one or more pads 220 to establish and maintain an intimate thermal contact between the pads 220 and the patient's skin, i.e., secure the pads to the patient. For example, a fastening device 236 in the form of a strap may be wrapped around a thigh of the patient so that a tension force of the strap exerts the downward force on the pad 220 applied to the thigh.

In the illustrated embodiment, the pads 220 include four individual pads 220A-220D. Pads 220A, 220B are configured (i.e., positioned and sized) for placement on the patient's torso. In some embodiments, each of the pads 220A, 220B are configured for placement adjacent each other on a front side of the torso. The pads 220A, 220B may also be configured to extend around a side of the torso to a back side of the torso. Similarly, the pads 220 include pads 220C, 220D configured (i.e., positioned and sized) for placement on and extension around each leg (e.g., thigh) of the patient 50.

In some embodiments, the contact system 210 may include FDL segments 203A extending between the pads 220 to provide for the flow of TTM fluid 102 to and from each of the pads 220. In some embodiments, any or all of the pads 220 may individually include an FDL 203 that extends between the pad 220 and the system module 105.

Although not required, an FDL segment 203A may be fluidly coupled with a corresponding pad 220 via a set of connectors 223 so that the FDL segment 203A may be selectively connected to and disconnected from the pad 220. The connectors 223 may facilitate omitting the use of any pad 220 for a given TTM therapy. The connectors 223 may also facilitate the replacement of a subset of the pads 220. For example, a pad of the gown assembly 215 may deemed unfit for further use (e.g., defective). In such an instance, the clinician may disconnect the defective pad and replace it with another pad.

Although not required, in some embodiments, the container 241 of the hydrogel source 240 may be configured to facilitate application of the hydrogel 245 to the pads 220. For example, the container 241 may facilitate spraying the hydrogel 245 onto an underside of the pads 220. In other embodiments, the clinician may remove a portion of the hydrogel 245 from the container 241 and apply the hydrogel 245 manually to the underside of the pads 220.

The pad 220 is secured to the panel 231 via any suitable fastening technique. In some embodiments, the pad 220 may be fixedly (i.e., permanently) attached to the gown 230. For example, the pad 220 may be attached to the gown 230 via permanent fasteners (e.g., rivets) disposed along the perimeter of the pad 220. In other embodiments, the pad 220 may be sewn to the panel 231. In still other embodiments, the pad 220 (or more specifically the top film 225) may be adhesively attached to the panel 231. In further embodiments, the pad 220 may be coupled with the panel via injection molding of a portion of the pad 220.

In other embodiments, the pad 220 may be removably attached to the gown 230. For example, the pad 220 may be attached to the panel 231 via non-permanent fasteners, such as snaps, buttons, or hooks, for example. The gown 230 may be formed of a fabric configured for repeated sterilization via exposure to an elevated temperature, such as via autoclaving, for example.

FIG. 2C is a cross-sectional view of a portion of the gown assembly 215 cut along sectioning lines 2C-2C. Shown is a cross section of the pad 220C which may be representative of any or all of the pads 220. The pads 220 include a top film 225 and a bottom film 226. The top film 225 is sealably coupled with bottom film 226 to form a fluid compartment 224 for containing the TTM fluid 102.

Either or both of the films 225, 226 may be formed of a flat sheet or may include a preformed shape. The films 225, 226 may be formed of any suitable material such as a plastic, silicone, rubber, and the like. The films 226, 226 may be flexible to define a flexibility of the pad 220 to accommodate placing the pad 220 along a not flat portion of the patient body, such around the leg, for example. The material of the films 225, 226 may be selected to allow for repeated disinfection and/or sterilization of the pad 220. In some embodiments, the films 225, 226 may accommodate repeated sterilization via autoclaving.

The pad 220 include an internal support structure 227. The support structure 227 may ensure a minimum separation distance between the films 225, 226 across the pad 220. The support structure 227 may prevent a crushing or flattening of the fluid compartment 224. The support structure 227 may also define fluid channels within the pad 220. In some embodiments, the support structure 227 may be attached to one or both of the films 225, 226.

A method of using the thermal contact system 210 to perform a TTM therapy may include all or any subset of the flowing steps or processes. The clinician may apply a layer of hydrogel 245 to the underside of the pads 220. Applying the hydrogel 245, may include spraying the hydrogel 245 on the underside of the pads 220. The clinician may place the thermal gown assembly 215 on a patient 50. The clinician may place one or more of the pads 220 on the patient at predefined locations of the patient 50. In some embodiments, the clinician may secure one or more pads 220 to the patient via the fastening device(s). In some embodiments, the clinician may omit the use of one or more pads 220 of the gown assembly 215 and in some embodiments, omitting the use of one or more pads 220 may include fluidly disconnecting the one or more pads 220 from the remaining pads 220. The clinician may couple the FDL 203 of the thermal contact system 210 to the system module 105 and initiate a flow of TTM fluid 102 through the pads 220 to perform a first TTM therapy. In some embodiments, the clinician may replace one or more of pads 220 of the gown assembly 215 during the first TTM therapy. At the conclusion of the first TTM therapy, the clinician may remove the gown assembly 215 from the patient. In some embodiments, the TTM therapy may be a first TTM therapy and the patient may be a first patient.

The clinician may then prepare the gown assembly 215 for a second TTM therapy. Preparing the gown assembly 215 may include removing the hydrogel 245 from the pads 220. In some embodiments, the clinician may launder the gown assembly 215 which may include disinfecting the pads 220. In some embodiments, the clinician may sterilize the gown assembly 215, and the sterilizing may include exposing the gown assembly 215 (or portions thereof) to an elevated temperature sufficient to cause sterilization. In some embodiments, the clinician may remove and/or replace one or more pads 220 of the gown assembly 215. The clinician may then perform the second TTM therapy utilizing the same gown assembly 215 in accordance with all or a subset of the processes described above for the first TTM therapy. In some embodiments, the clinician may perform the second TTM therapy on a second patient different from the first patient.

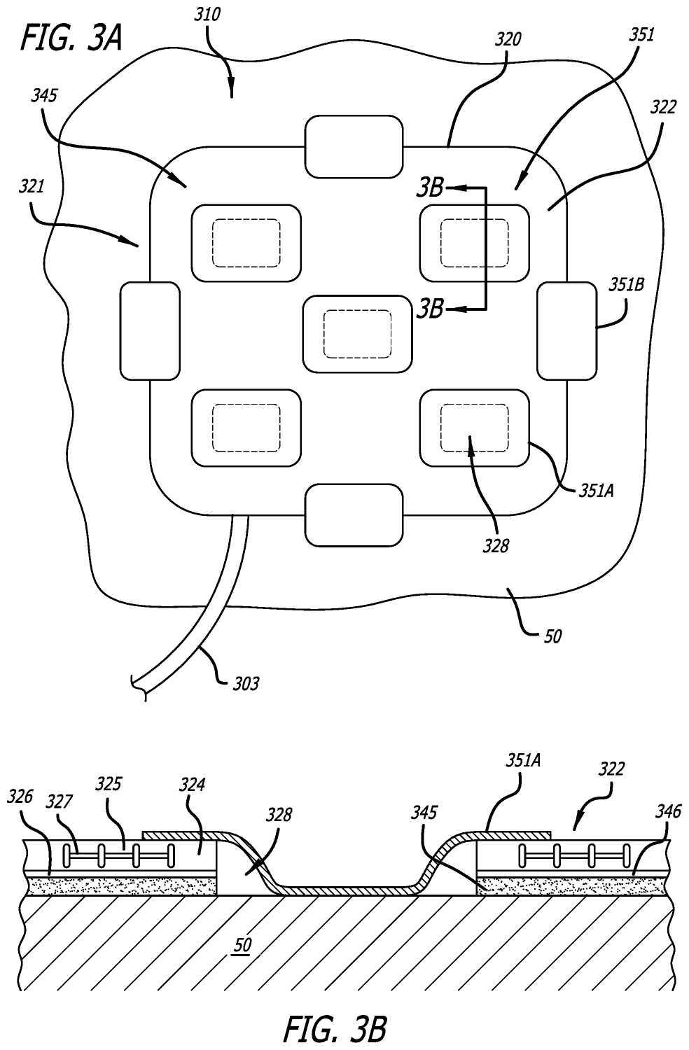

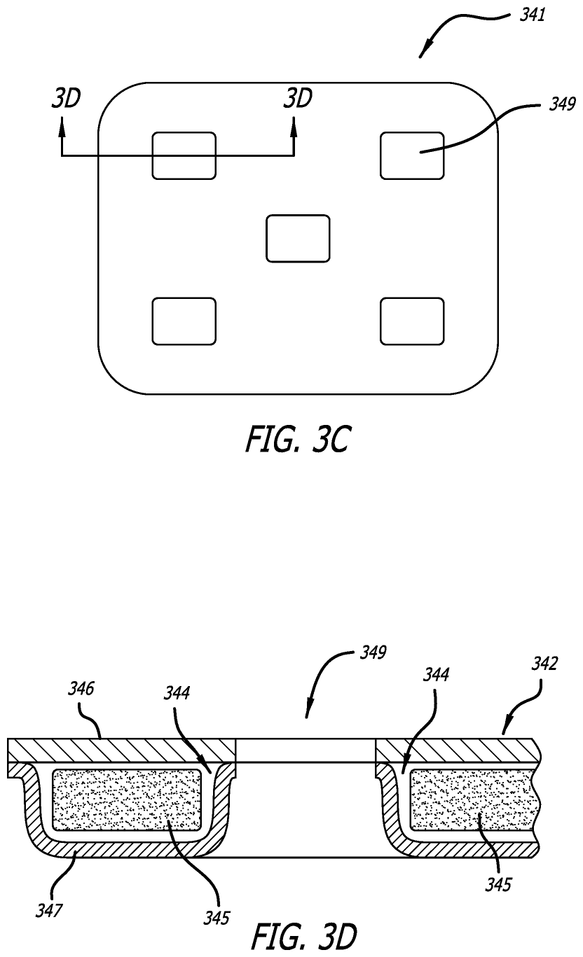

FIGS. 3A-3D provide various illustrations of a second embodiment of a thermal contact system 310 that can, in certain respects, resemble components of the thermal contact system 210 described in connection with FIGS. 2A-2C. It will be appreciated that all the illustrated embodiments may have analogous features. Accordingly, like features are designated with like reference numerals, with the leading digits incremented to “3.” For instance, the fluid compartment is designated as “224” in FIGS. 2A-2C, and an analogous fluid compartment is designated “324” in FIGS. 3A-3D. Relevant disclosure set forth above regarding similarly identified features thus may not be repeated hereafter. Moreover, specific features of the thermal contact system 210 and related components shown in FIGS. 2A-2C may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the thermal contact system 310 of FIGS. 3A-3D. Any suitable combination of the features, and variations of the same, described with respect to the thermal contact system 210 and components illustrated in FIGS. 2A-2C can be employed with the thermal contact system 310 and components of FIGS. 3A-3C, and vice versa. This pattern of disclosure applies equally to further embodiments depicted in subsequent figures and described hereafter.

FIG. 3A is a top view illustration of the thermal contact system (system) 310 that may be employed with the TTM system 100 of FIG. 1. In some embodiments, a plurality of contact systems 310 may be employed with the TTM system 100. FIG. 3A illustrates the thermal contact system 310 applied to a patient 50. The thermal contact system 310 generally includes a thermal contact pad 320, a plurality of adhesive patches 351, and a hydrogel 345 disposed on an underside 321 of the pad 320 so as to be disposed in contact with the patient 50 (i.e., the patient's skin). In some embodiments, the adhesive patches 351 may be sterilized. The thermal contact system 310 includes a fluid delivery line 303 for fluid coupling of the pad 320 with the TTM system module 105 (see FIG. 1).

The thermal contact system 310 may be configured for multiple uses with a single patient or multiple uses across multiple patients. The pad 320 may be configured for repeated cleaning, including disinfection and/or sterilization. In some embodiments, the pad 320 may be configured for sterilization via autoclaving.

The pad 320 includes a plurality of openings 328 extending between a topside 322 and the underside 321 of the 320. The openings 328 may facilitate visual inspection of the patient's skin during the TTM therapy without separating the pad 320 from the patient 50.

The adhesive patches 351 are utilized to secure the pad 320 to the patient 50. A first subset of adhesive patches 351A are utilized secure the pad 320 to the patient 50 at the openings 328 as further described below. Another subset of the adhesive patches 351B are utilized to secure the pad 320 to the patient 50 along a perimeter edge of the pad 320.

FIG. 3B is a cross-sectional view a portion of the system 320 cut along sectioning lines 3B-3B with pad 320 secured to the patient 50. The pad 320 includes a fluid compartment 324 sealed between a top film 325 and bottom film 326. Either or both of the films 325, 326 may be formed of a flat sheet or include a preformed shape. The films 325, 326 may be formed of any suitable material such as a plastic, silicone, rubber, and the like. The films 326, 326 may be flexible to define a flexibility of the pad 320 to accommodate placing the pad 320 along a not flat portion of the patient body, such as the leg, for example. The material of the films 325, 326 may be selected to allow repeated disinfection and/or sterilization of the pad 320. In some embodiments, the films 325, 326 may accommodate repeated sterilization.

The pad 320 may include an internal support structure 327. The support structure 327 may ensure a minimum separation distance between the films 325, 326 across the pad 320. The support structure 327 may prevent a crushing or flattening of the fluid compartment 324. The support structure 327 may also define fluid channels within the fluid compartment 324. In some embodiments, the support structure 327 may be attached to one or both of the films 325, 326.

The hydrogel 345 is disposed on the underside 321 of the pad 320 to define an intimate thermal contact with the patient 50. The hydrogel 345 does not extend across the openings 328. In some embodiments, the hydrogel 345 may be coupled with a hydrogel liner 346 disposed between the hydrogel 345 and the bottom film 326. The hydrogel liner 346 may be removably attached to the bottom film 326 (e.g., via an adhesive). In some embodiments, the hydrogel liner 346 may prevent direct contact of the hydrogel 345 with the pad 320. In some embodiments, the hydrogel 345 may be separate from (i.e., not coupled with) the pad 320 when pad 320 is not applied to the patient 50 as further described below. In some embodiments, the hydrogel liner 346 may be omitted.

The adhesive patch 351A secures the pad 320 to the patient 50 through the opening 328. As illustrated embodiment, the adhesive patch 351 overlaps the topside 322 of the pad 320 along the perimeter edge of the opening 328. In some embodiments, the adhesive patch 351 may overlap the topside 322 along only portions of the perimeter edge such as along opposite sides of the opening 328. In other embodiments, the adhesive patch 351 may overlap the topside 322 along an entire circumferential edge of the opening 328. A central portion of the adhesive patch 351A is depressed through the opening 328 so as to contact and adhere to the skin of the patient 50 via an adhesive disposed on an underside of the adhesive patch 351A. In use, the clinician may separate all or a portion of the adhesive patch 351a from the pad 320 to enable inspection of the patient's skin through the opening 328.

FIGS. 3C-3D illustrate an exemplary hydrogel container 341 that, although not required, in some embodiments, may be included in the system 310. FIG. 3C is a top view of the container 341 and FIG. 3D is a cross-sectional view of a portion of the container 341. In some instances, a hydrogel may generally dry out overtime when not sealed within a container. The container 341 prevents dehydration of the hydrogel 345. As discussed above, the hydrogel 345 may be applied to the bottom film 326 prior to application of the pad 320 to the patient 50. The hydrogel container 341 is configured to facilitate application of the hydrogel 345 to the pad 320 so as to prevent the hydrogel 345 from encroaching on the openings 328.

The hydrogel container 341 is shaped similar to the pad 320 and includes container openings 349 that align with the openings 328 of the pad 320. The hydrogel container 341 includes a bottom liner 347 sealably coupled with the hydrogel liner 346 along the outside circumferential edge of the hydrogel container 341 and along the circumferential edge of each container opening 349 to define a hydrogel compartment 344. The bottom liner 347 is removably attached to the hydrogel liner 346 so that the clinician may separate the bottom liner 347 from the hydrogel liner 346 exposing the hydrogel 345. An adhesive 342 is disposed along the topside of the hydrogel liner 346 to facilitate removable attachment of the hydrogel container 341 to the pad 320 (i.e., the bottom film 326).

The hydrogel container 341 is illustrated as a single container holding all the hydrogel 345. In other embodiments, the hydrogel 345 may be divided into multiple hydrogel containers. In other words, the hydrogel container 341 may include multiple separate pouches to be attached to the pad 320. In some embodiments, the pouches may be shaped into strips.

As the thermal contact system 310 is configured for multiple uses, i.e., multiple applications of the pad 320 to one or more patients, and as the hydrogel 345 is configured for a single use, the thermal contact system 310 may include multiple hydrogel containers 341 so that a first hydrogel container 341 may be replaced with a second hydrogel container 341 between uses of the pad 320.

A method of using the thermal contact pad system 310 to perform a TTM therapy may include all or any subset of the flowing steps or processes. The clinician may apply the hydrogel 345 to the underside of the pad 320. Applying the hydrogel 345, may include coupling the hydrogel container 341 with the underside of the pad 320. Applying the hydrogel 345, may further include removing a bottom liner 347 from the hydrogel liner 346 to expose the hydrogel 345. The clinician may apply the pad 320 to the patient at a predefined location of the patient 50. The clinician may then secure the pad 320 to the patient 50 by placing one or more adhesive patches along the circumferential edge of the pad 320. The clinician may further secure the pad 320 by depressing adhesive patches through any or all openings of the pad 320. The clinician may couple the FDL 303 of the system 310 to the system module 105 and initiate a flow of TTM fluid 102 through the pad 320 to perform a first TTM therapy. At the conclusion of the first TTM therapy, the clinician may remove the pad 320 from the first patient.

The clinician may then prepare the pad 320 for a second TTM therapy. Preparing the pad 320 may include removing the hydrogel 345 from the pad 320 which may include separating the hydrogel liner 346 from the underside of the pad 320. The clinician may then perform the second TTM therapy utilizing the same pad 320 in accordance with all or a subset of the processes described above for the first TTM therapy. In some embodiments, the clinician may perform the second TTM therapy on a second patient different from the first patient.

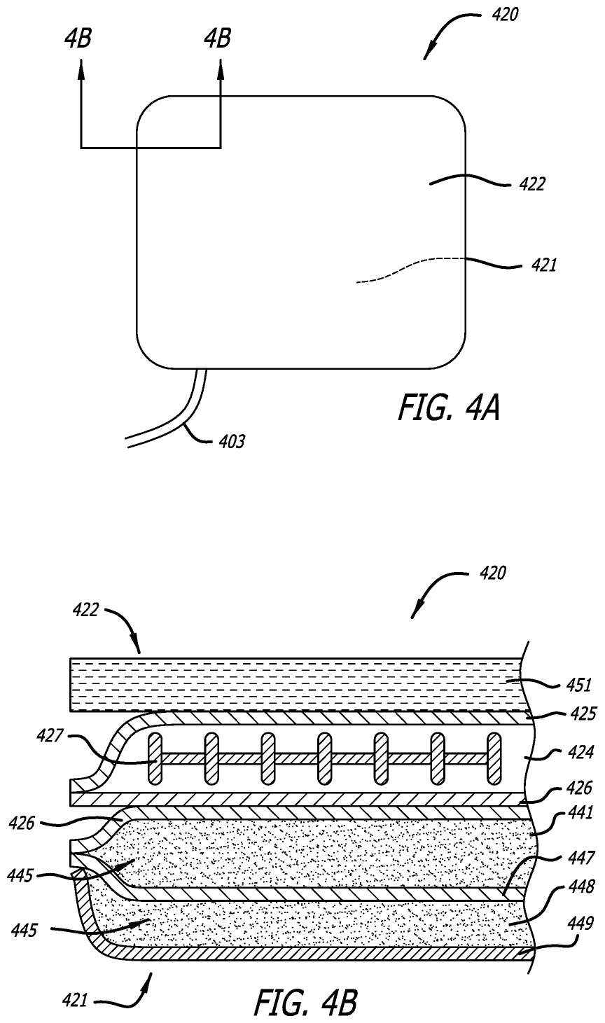

FIG. 4A is a top view of the thermal contact pad 420 that may be employed with the TTM system 100 of FIG. 1. In some embodiments, a plurality of pads 420 may be employed with the TTM system 100. The pad 420 includes a fluid delivery line 403 for fluid coupling of the pad 420 with the TTM system module 105 (see FIG. 1). The pad 420 may be configured for multiple uses with a single patient or multiple uses across multiple patients. The pad 420 includes multiple layers disposed between a topside 422 and underside 421.

FIG. 4B is a cross-sectional view a portion of the pad 420 cut along sectioning lines 4B-4B. The pad 420 includes a fluid compartment 424 sealed between a top film 425 and bottom film 426, i.e., the top film 425 may be sealably coupled with the bottom file 426 along a circumferential perimeter edge of the pad 420. Either or both of the films 425, 426 may be formed of a flat sheet or include a preformed shape. The films 425, 426 may be formed of any suitable material such as a plastic, silicone, rubber, and the like. The films 426, 426 may be flexible to define a flexibility of the pad 420 to accommodate placing the pad 420 along a not flat portion of the patient body, such as the leg, for example.

The pad 420 may include an internal support structure 427 disposed within the fluid compartment 424. The support structure 427 may ensure a minimum separation distance between the films 425, 426 across the pad 420. The support structure 427 may prevent a crushing or flattening of the compartment 424. The support structure 427 may also define fluid channels within the pad 420. In some embodiments, the support structure 427 may be attached to one or both of the films 425, 426.

The pad 420 include multiple layers of hydrogel 445 to facilitate multiple uses of the pad 420. In the illustrated embodiment, the pad 420 includes two hydrogel layers, i.e., a first hydrogel layer 441 and a second hydrogel layer 448. However, in other embodiments, the pad 420 may include more than 2 hydrogel layers. Each hydrogel layer is configured to contact the patient's skin directly to define an intimate thermal contact with the patient 50.

The hydrogel layers are coupled with the fluid compartment 424 so as to facilitate thermal energy exchange with the patient. The first hydrogel layer 441 is disposed adjacent the fluid compartment 424. Although not required, in some embodiments, a first hydrogel liner 446 may be disposed between the hydrogel layer 441 and the bottom film 426. In some embodiments, the first hydrogel liner 446 may be omitted. Disposed along an underside of the first hydrogel layer 441 is a second hydrogel liner 447. The second hydrogel liner 447 may be sealably and removably coupled with either the first hydrogel liner 446 or the bottom film 426 along a circumferential edge of the pad 420 to encapsulate the hydrogel 445 disposed within the first hydrogel layer 441, thereby preventing the hydrogel 445 within the first hydrogel layer 441 from drying out or otherwise degrading.

The second hydrogel layer 448 is disposed along an underside of the second hydrogel liner 447. A third hydrogel liner 449 may be removably coupled with the second hydrogel liner 447, the first hydrogel liner 446, or the bottom film 426 along the circumferential edge of the pad 420 to encapsulate the hydrogel 445 disposed within the second hydrogel layer 448, thereby preventing the hydrogel 445 within the second hydrogel layer 448 from degrading.

Although not required, in some embodiments, the pad 420 may include a foam layer 451 disposed along a topside of the fluid compartment 424. In some embodiments, the foam layer 451 may be formed of a polychloroprene material, such as Neoprene, for example.

In some embodiments, the hydrogel 445 may be configured to provide an indication to the clinician that one or more operating properties (e.g., flexibility, thermal conductivity, adhesion, or hydration) have sufficiently degraded to warrant replacement. According to one example, the hydrogel 445 may be configured to change color during use to indicate that replacement of the hydrogel 445 may be advantageous. By way of another example, the hydrogel 445 may include electrical intelligence, such as an embedded circuit (not shown) to provide the visual indication (e.g., LED illumination or darkening). In some embodiments, the hydrogel 445 may be compostable and/or biodegradable.

A method of using the pad 420 to perform a TTM therapy may include all or any subset of the flowing steps or processes. The clinician may separate the third hydrogel liner 449 from the pad 420 to expose the second hydrogel layer 448. The clinician may apply the pad 420 to the patient at a predefined location of the patient 50 so that the second hydrogel layer 448 defines an intimate thermal contact with the patient. The clinician may couple the FDL 403 of the pad 420 to the system module 105 and initiate a flow of TTM fluid 102 through the pad 420 to perform a first TTM therapy. At the conclusion of the first TTM therapy, the clinician may remove the pad 420 from the first patient.

The clinician may then prepare the pad 420 for a second TTM therapy. Preparing the pad 420 may include removing the second hydrogel layer from the pad 420 and separating the second hydrogel liner 447 from the pad 420 to expose the hydrogel 445 of the first hydrogel layer 441. The clinician may then apply the pad 420 to the patient at a predefined location of the patient 50 so that the first hydrogel layer 448 defines an intimate thermal contact with the patient. The clinician may couple the FDL 403 of the pad 420 to the system module 105 and initiate a flow of TTM fluid 102 through the pad 420 to perform a second TTM therapy.

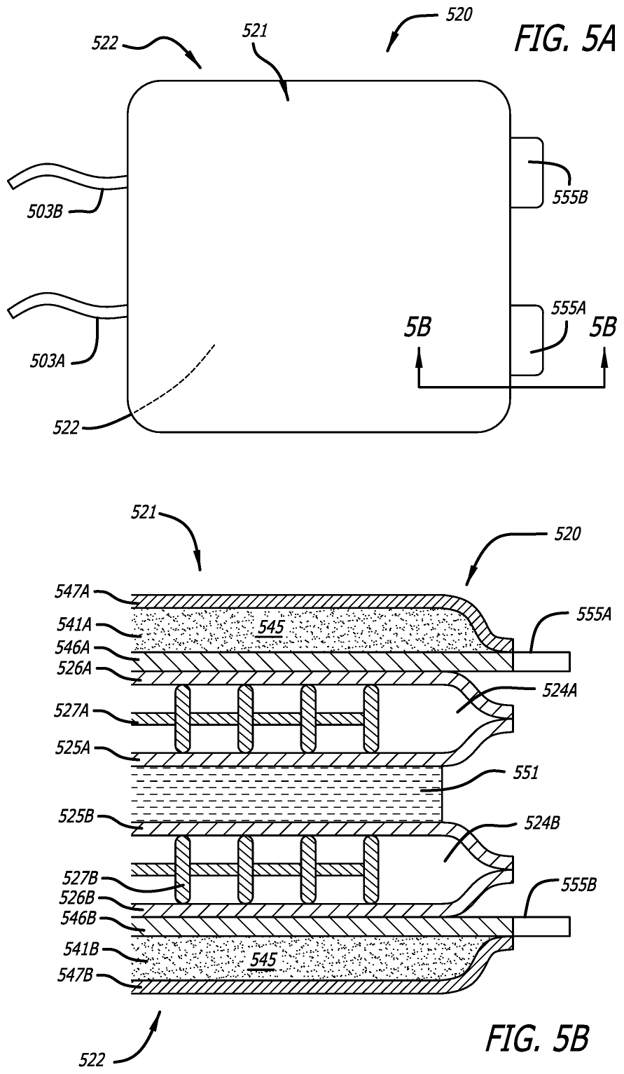

FIG. 5A is a top view of the thermal contact pad 520 that may in some respects resemble the pad 420 shown and described above. FIG. 5B is a cross-sectional view a portion of the pad 520 cut along sectioning lines 5B-5B. The thermal contact pad 520 that may be employed with the TTM system 100 of FIG. 1 to perform a TTM therapy. In some embodiments, a plurality of pads 520 may be employed with the TTM system 100. The pad 520 is configured for multiple uses with a single patient or multiple uses across multiple patients. The pad 520 is configured for defining a thermal contact with the patient when the pad 520 is disposed in a first orientation and/or a second orientation. In the first orientation, the pad 520 is applied to the patient so that a first side 521 faces toward the patient and second side 522 faces away from the patient. In the second orientation, the pad 520 the second side 522 faces toward the patient and first side 521 faces away the patient.

The pad 520 includes multiple layers disposed between the first side 521 and second side 522. The pad 520 includes a foam layer 551 which may be disposed centrally between the first side 521 and second side 522. In some embodiments, the foam layer 551 may be formed of a polychloroprene material, such as Neoprene, for example.

The pad 520 includes a first set of layers disposed between the foam layer 551 and the first side 521. The pad 520 includes a first fluid compartment 524A for containing the TTM fluid 102 (FIG. 1) and circulating of the TTM fluid 102 therein. The first fluid compartment 524A is sealed between a first inner film 525A and first outer film 526A. The pad 520 includes a first fluid delivery line 503A coupled with the first fluid compartment 524A to facilitate flow of TTM fluid 102 between the first fluid compartment 524A and the TTM system module 105 (see FIG. 1). The first fluid compartment 524A may include a first support structure 527A disposed therein. The first support structure 527A may ensure a minimum separation distance between the films 525A, 526A across the pad 520. The first support structure 527A may prevent a crushing or flattening of the compartment 524A. The first support structure 527A may also define fluid channels within the first fluid compartment 524A. In some embodiments, the first support structure 527A may be attached to one or both of the films 525A, 526A.

The pad 520 includes first hydrogel layer 541A coupled with the first fluid compartment 524A opposite the foam layer 551. In some embodiments, a first hydrogel liner 546A may be disposed between the first hydrogel layer 541A and the first outer film 526A. The first hydrogel liner 546A may include a first pull tab 555A to facilitate separation/removal of the first hydrogel liner 546A from the pad 520. Removal of the first hydrogel liner 546A may include removal of the first hydrogel layer 541A therewith.

Although not required, in some embodiments, a first storage liner 547A may be disposed along an outerside of the first hydrogel layer 541A. The first storage liner 547A may be removably coupled with either the first hydrogel liner 546A or the first outer film 526A along a circumferential edge of the pad 520 to sealably encapsulate the first hydrogel layer 541A, thereby preventing the hydrogel 545 within the first hydrogel layer 541A from drying out or otherwise degrading over time.

The pad 520 also includes a second set of layers disposed between the foam layer 551 and the second side 522. The layers disposed between the foam layer 551 and the second side 522 may resemble the first set of layers disposed between the foam layer 551 and the first side 521. As such, the reference numbers of the first set of layers are designated with an “A” suffix and the second set of layers are designated with a “B” suffix. As shown the second set of layers are arranged symmetrically in relation to the first set of layers.

One or more of the layers of the pad 520 shown and described may be optional, i.e., not required. In some embodiments, the foam layer 551 may be omitted and in such embodiments, either the first fluid compartment 524A or the second fluid compartment 524B may also be omitted.

In some embodiments, the first and second fluid compartments 524A, 524B may be fluidly coupled together. In such embodiments, a single FDL may facilitate flow of the TTM fluid 102 through both of the first and second fluid compartments 524A, 524B. As such, either the first FDL 603A or the second FDL 603B may be omitted.

A method of using the pad 520 to perform a TTM therapy may include all or any subset of the flowing steps or processes. The clinician may separate the first storage liner 547A from the pad 520 to expose the first hydrogel layer 541A. The clinician may apply the pad 520 to a patient at a predefined location of the patient 50 so that the first hydrogel layer 541A defines a thermal contact with the patient. The clinician may couple the first FDL 503A of the pad 520 to the system module 105 and initiate a flow of TTM fluid 102 through the first fluid compartment 524A to perform a first TTM therapy. At the conclusion of the first TTM therapy, the clinician may remove the pad 520 from the patient.

The clinician may prepare the pad 520 for a second TTM therapy. The clinician may remove the first hydrogel layer 541A from the pad 520. The clinician may also remove the first hydrogel liner 546A from the pad 520. The clinician may remove the second storage liner 547B from the pad 520 to expose the second hydrogel layer 541B. The clinician may then apply the pad 520 to a patient at a predefined location of the patient 50 in an inverted orientation with respect to the orientation of the pad during the first TTM therapy so that the second hydrogel layer 541B defines a thermal contact with the patient. The clinician may couple the second FDL 503B of the pad 520 to the system module 105 and initiate a flow of TTM fluid 102 through the second fluid containing layer 524B to perform the second TTM therapy. At the conclusion of the second TTM therapy, the clinician may remove the pad 520 from the patient. The first and second TTM therapies may be performed on the same or different patients.

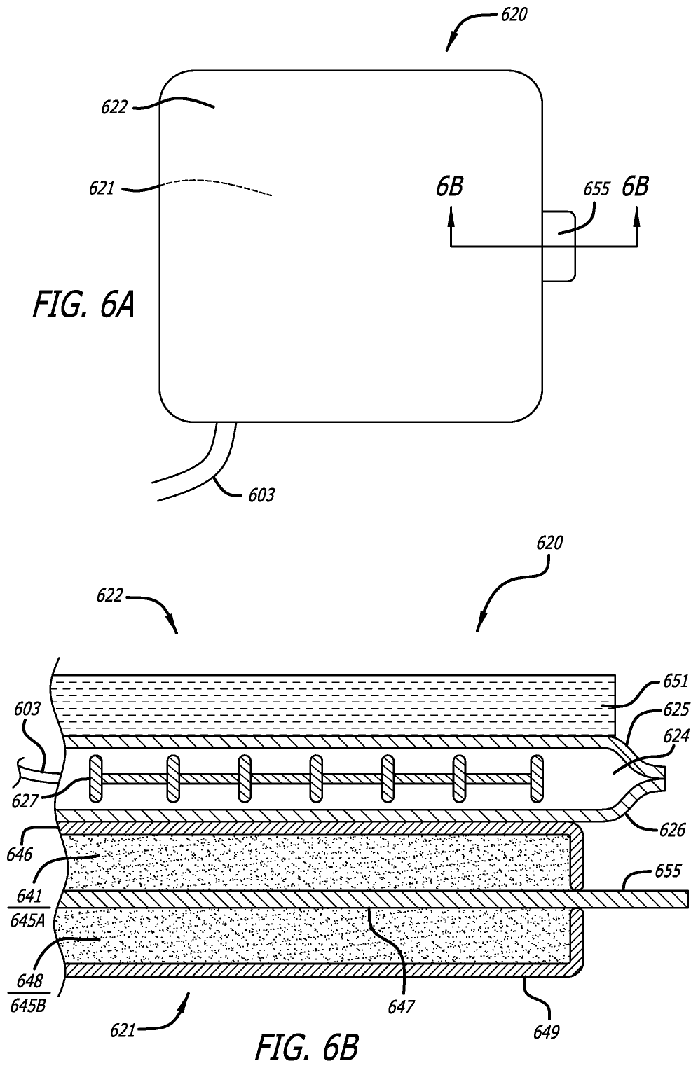

FIG. 6A is a top view of the thermal contact pad 620 that may be employed with the TTM system 100 of FIG. 1. In some embodiments, a plurality of pads 620 may be employed with the TTM system 100. The pad 620 includes a fluid delivery line 603 for fluid coupling of the pad 620 with the TTM system module 105 (see FIG. 1). The pad 620 include multiple layers disposed between a topside 622 and underside 621. The pad 620 is configured for extended use. In some current instances, the hydrogel of a thermal contact pad may dry out before the completion of a TTM therapy which may require replacement of the pad to complete the therapy. The pad 620 is configured to extend the useful life of a hydrogel layer disposed on the underside 221 of the pad 620 when the pad 620 is applied to the patient. The pad 620 includes a pull tab 655 configured to cause an extended hydration of a hydrogel layer of the pad 620 when the pull tab 655 is pulled by the clinician as further described below.

FIG. 6B is a cross-sectional view a portion of the pad 620 cut along sectioning lines 6B-6B. The pad 620 includes a fluid compartment 624 sealed between a top film 625 and bottom film 626. The pad 620 may include an internal support structure 627 disposed within the fluid compartment 624. The support structure 627 may ensure a minimum separation distance between the films 625, 626 across the pad 620. The support structure 627 may prevent a crushing or flattening of the compartment 624. The support structure 627 may also define fluid channels within the pad 620. In some embodiments, the support structure 627 may be attached to one or both of the films 625, 626.

In the illustrated embodiment, the pad 620 includes a foam layer 651 disposed along a topside of the fluid compartment 624. In some embodiments, the foam layer 651 may be formed of a polychloroprene material, such as Neoprene, for example. In some embodiments, the foam layer 651 may be omitted.

The pad 620 includes multiple layers of hydrogel. In the illustrated embodiment, the pad 620 include two hydrogel layers, i.e., a first hydrogel layer 641 and a second hydrogel layer 648. However, in other embodiments, the pad 620 may include more than two hydrogel layers. The second hydrogel layer 648 is configured to contact the patient's skin directly to define the thermal contact with the patient 50.

The hydrogel layers 641, 648 are coupled with the fluid compartment 624 to facilitate thermal energy exchange between the fluid compartment 624 and the patient. The first hydrogel layer 641 is disposed adjacent the fluid compartment 624. Although not required, in some embodiments, a first hydrogel liner 646 may be disposed between the hydrogel layer 641 and the bottom film 626. In some embodiments, the first hydrogel liner 646 may be omitted.

Disposed along an underside of the first hydrogel layer 641 is a separation liner 647. The separation liner 647 may be removably coupled either the first hydrogel liner 646 or the bottom film 626 along a circumferential edge of the pad 620 to encapsulate the hydrogel 645 disposed within the first hydrogel layer 641.

The second hydrogel layer 648 is disposed along an underside of the second hydrogel liner 647. The pad 620 may include a third hydrogel liner 649 removably coupled with the separation liner 647, the first hydrogel liner 646, or the bottom film 626 along the circumferential edge of the pad 620 to encapsulate the hydrogel 645 disposed within the second hydrogel layer 648. The separation liner 647 is disposed between the first hydrogel layer 641 and second hydrogel layer 648 to define an isolation of the first hydrogel layer 641 from the second hydrogel layer 648.

The pull tab 655 is attached to (or integrally part of) the separation liner 647. The pull tab 655 extends outward of a perimeter edge of the pad 620 to provide for manual access of the tab 655 by the clinician. The pad 620 is generally configured so that pulling the pull tab 655 removes the isolation between the first hydrogel layer 641 and the second hydrogel layer 648. Removing the isolation may include removing the separation liner 647 (or a portion thereof) from between the first hydrogel layer 641 and the second hydrogel layer 648. Removing the isolation provides for a migration of one or more constituents (e.g., water) of the first hydrogel layer 641 into the second hydrogel layer 648.

Generally speaking, the second hydrogel layer 648 is configured for direct contact with the patient's skin to define a thermal contact with the patient, and the first hydrogel layer 641 is configured to extend the operational life of the second hydrogel layer 648 upon removal of the separation liner 647. In some embodiments, the first hydrogel layer 641 is configured to rehydrate (or extend the hydration of) upon removal of the separation liner 647.

In some embodiments, the first hydrogel layer 641 may include a hydrogel 645A, and the second hydrogel layer 648 may include a hydrogel 645B that is different than the hydrogel 645A. For example, the second hydrogel 645B may include an ultraviolet light-cured composition that includes: (i) a cross-linking copolymer in an amount of between about 15% to 30% by weight of said composition, (ii) water in an amount of between about 15% to 40% by weight of said composition, and (iii) glycerol in an amount of between about 25% to 35% by weight of the composition. In some embodiments, the first hydrogel 645A may include a composition having similar constituents but having a greater relative amount of water than the second hydrogel 645B. In other embodiments, the hydrogels 645A, 648B may be the same.

In some embodiments, one or both the hydrogels 645A, 648B may be configured to provide an indication to the clinician that one or more operating properties (e.g., flexibility, thermal conductivity, adhesion, or hydration) have sufficiently degraded such as described above in relation the hydrogel 445 of FIG. 4B. For example, the hydrogel 645B may provide an indication to the clinician that the hydrogel 645B is sufficiently dehydrated to warrant pulling the pull tab 655 to initiate the rehydration of the hydrogel 645B.

A method of using the pad 620 to perform a TTM therapy may include all or any subset of the flowing steps or processes. The clinician may separate the third hydrogel liner 649 from the pad 620 to expose the second hydrogel layer 648. The clinician may apply the pad 620 to the patient at a predefined location of the patient 50 so that the second hydrogel layer 648 defines an intimate thermal contact with the patient. The clinician may couple the FDL 603 of the pad 620 to the system module 105 and initiate a flow of TTM fluid 102 through the pad 620 to perform a TTM therapy.

The clinician may then prepare the pad 620 for extended use. Preparing the pad 620 may include removing the separation liner 647 (or a portion thereof) from between the first hydrogel layer 641 and the second hydrogel layer 648 of the pad 620 so that the hydrogel 645A may rehydrate (or extend the hydration of) the second hydrogel 645B within the second hydrogel layer 648. In some embodiments, removing the separation liner 647 may include pulling the pull tab 655. In some embodiments, the clinician may pull the pull tab 655 to remove the separation liner 647 while the pad 620 is applied to the patient. In further embodiments, the clinician may pull the pull tab 655 to remove the separation liner 647 while the TTM fluid 102 (see FIG. 1) is flowing through the pad 620.

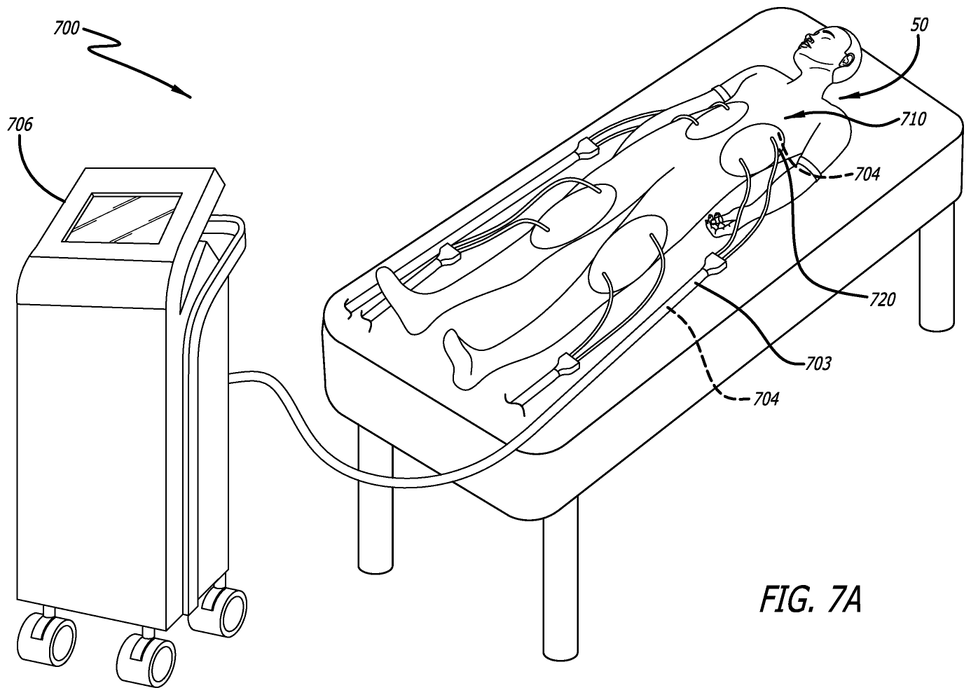

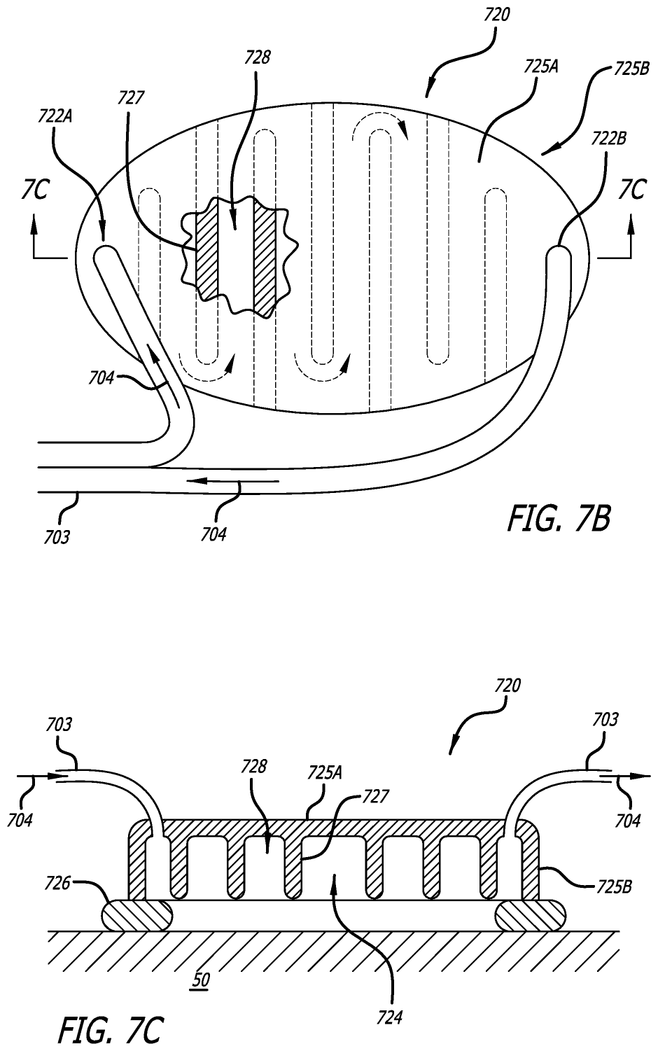

FIG. 7A illustrates another embodiment of a targeted temperature management (TTM) system 700 connected to a patient 50 for administering a TTM therapy to the patient 50 which may include a cooling and/or warming of the patient 50, in accordance with some embodiments. The TTM system 700 includes a TTM system module 705 for preparing and delivering TTM fluid (e.g., air) 704 to a thermal contact system 710 which may, in some embodiments, include one more thermal contact pads 720. The TTM system 700 includes one or more bidirectional fluid deliver lines (FDL) 703 extending from the TTM module 105 to the pads 720 to provide for flow of TTM air 704 between the TTM module 705 and the pads 720.

In use, the TTM module 705 prepares the TTM air 704 for delivery to the pad 720 by heating or cooling the TTM air 704 to a defined temperature in accordance with a prescribed TTM therapy. The TTM module 705 circulates the TTM air 704 within/through the pads 720 to facilitate thermal energy exchange with the patient 50. During the TTM therapy, the TTM module 705 may continually control the temperature of the TTM air 704 toward a target TTM temperature. As shown, the pad(s) 720 may be applied to different body parts of the patient 50. As such, the pads 720 may be available in different configurations, such as sizes and shapes, for example, to accommodate the different body parts. The TTM module 705 delivers TTM air 704 to the pads 720 so that the pressure of the air TTM 704 is negative (i.e., a vacuum).

FIG. 7B is a top view of an exemplary pad 720 and the FIG. 7C is a cross-sectional side view of the pad 720 applied to the patient 50. The pad 720 includes a top wall 725A coupled with a circumferential side wall 725B extending along the circumference of the pad 720. A circumferential sealing member 726 extends a long a bottom edge of the side wall 725B to form a fluid/air seal with the patient 50. The pad 720 includes a flexibility so that the pad 720 may conform to (i.e., match curvatures of) uneven surfaces of the patient 50. The sealing member 726 also includes a flexibility to extend over protrusions and/or within depressions of the patient 50 as to form the fluid tight seal. In some embodiments, the flexibility of the sealing member 726 exceeds the flexibility of the top wall 725A and the side wall 725B. The top wall 725A, the side wall 725B, and the sealing member 726 combine with the patient's skin to form compartment 724 for containing the air 704. In use, the pressure within the compartment 724 is a vacuum so that atmospheric pressure defines a downward force 708 of the pad 720 toward/onto the patient 50.

Although not required, the pad 720 may include a support structure 727 within the compartment 724 that prevents crushing of the compartment 724 due to the downward force 708 or any other external force. The support structure 727 may also define flow channels 728 for the air 704 to flow within the compartment 724 between an inlet port 722A and an outlet port 722B. The support structure 727 may be coupled with the top wall 725A. In use, the air 704 flows adjacent the skin of the patient 50 so that a temperature difference between the air 704 and the patient 50 defines a thermal energy exchange with the patient 50. The top wall 725A and the side wall 725B may be formed of any suitable material, such as rubber, silicone, ethylene propylene diene terpolymer (EPDM), or a flexible thermoplastic, for example. The sealing member 726 may be formed of similar materials to the top wall 725A and side wall 725B further including a foam material, such as neoprene, for example.

A method of using the TTM system 700 to perform a TTM therapy may include all or any subset of the flowing steps or processes. The clinician may apply the pad 620 to the patient at a predefined location of the patient 50. The clinician may initiate delivery of the TTM air 704 to the pad 720 at a negative pressure so that downward force 708 of the atmospheric pressure may secure the pad 720 to the patient. At the conclusion of the TTM therapy, the clinician may cease the delivery of the TTM air 704 to the pad 720 at the negative pressure so that the downward force 708 is eliminated. The clinician may remove the pad 720 from the patient. In some embodiments, the clinician may first remove the pad 720 from the patient and then cease the delivery of the TTM air 704. In alternative embodiments, the clinician may first cease the delivery of the TTM air 704 and then remove the pad 720 from the patient.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A patient contact system for exchanging thermal energy with a patient, the system comprising:
a gown assembly, comprising:
a gown configured for placement on the patient; and
a thermal contact pad coupled with the gown, the pad including a fluid compartment configured to receive a TTM fluid from a TTM system module via a fluid delivery line, the TTM system module configured to circulate the TTM fluid within the fluid compartment during the performance of a TTM therapy; and
a quantity of hydrogel applied to a patient contact side of the pad, wherein the quantity of hydrogel is dispensed from a hydrogel container.

2. The system of claim 1, wherein the system is configured for multiple uses on a single patient.

3. The system of claim 1, wherein the system is configured for multiple uses across different patients.

4. The system of claim 1, wherein the gown assembly is configured for disinfection.

5. The system of claim 1, wherein the gown assembly is configured for sterilization.

6. The system of claim 5, wherein the sterilization includes autoclaving.

7. The system of claim 1, wherein the gown assembly is configured for laundering within an automated laundering machine.

8. The system of claim 1, wherein the gown assembly comprises a plurality of thermal contact pads.

9. The system of claim 8, wherein the plurality of thermal contact pads comprises:
a first pad coupled with the gown so as to be located adjacent a first side of a torso of the patient when the gown assembly is placed on the patient, and
a second pad coupled with the gown so as to be located adjacent a second side of the torso opposite the first side when the gown assembly is placed on the patient.

10. The system of claim 8, wherein the plurality of thermal contact pads further comprises:
a third pad coupled with the gown so as to be located adjacent a first thigh of the patient when the gown assembly is placed on the patient, and
a fourth pad coupled with the gown so as to be located adjacent a second thigh of the patient when the gown assembly is placed on the patient.

11. The system of claim 8, wherein one or more of the plurality of thermal contact pads is removably coupled with the gown assembly.

12. The system of claim 8, wherein the plurality of thermal contact pads are fluidly interconnected.

13. The system of claim 12, wherein one or more of the plurality of thermal contact pads are fluidly disconnectable from the remaining pads.

14. The system of claim 8, wherein two or more of the plurality of thermal contact pads are individually coupled with the TTM system module.

15. The system of claim 1, wherein:
the gown assembly includes a plurality of fastening devices, and
one or more of the plurality of fastening devices are configured to secure the gown to the patient.

16. The system of claim 15, wherein one or more of the plurality of fastening devices are configured to secure one or more of the plurality of pads in thermal contact with the patient.

17. The system of claim 1, wherein the fluid compartment includes a support structure disposed therein to define a minimum thickness of the fluid compartment.

18. A method of providing one or more targeted temperature management (TTM) therapies to one or more patients, comprising:
applying a gown assembly to a first patient, the gown assembly comprising:
a gown configured for placement on a patient; and
a plurality of thermal contact pads coupled with the gown, each pad including a fluid compartment configured to receive a TTM fluid from a TTM system module via a fluid delivery line, the TTM system module configured to circulate the TTM fluid within the fluid compartment during the performance of the TTM therapy;
dispensing a hydrogel from a hydrogel container onto a patient contact side of each pad;
securing each pad in thermal contact with the first patient so that the hydrogel is disposed between each corresponding fluid compartment and the first patient;
coupling the gown assembly to the TTM system module; and
circulating the TTM fluid through the fluid compartment of each pad to perform a first TTM therapy.

19. The method of claim 18, further comprising:
removing the gown assembly from the first patient;
applying the gown assembly to a second patient;
dispensing the hydrogel from the hydrogel container onto each pad a second time;
securing each pad in thermal contact with the second patient; and
circulating the TTM fluid through the fluid compartment of each pad a second time.

20. The method of claim 19, wherein the first patient and the second patient are different patients.

21. The method of claim 19, further comprising disinfecting the gown assembly between the first TTM therapy and the second TTM therapy.

22. The method of claim 19, further comprising sterilizing the gown assembly between the first TTM therapy and the second TTM therapy.

23. The method of claim 19, further comprising laundering the gown assembly with an automated laundering machine between the first TTM therapy and the second TTM therapy.

24. The method of claim 18, further comprising:
decoupling a thermal contact pad from the gown assembly, and
coupling a replacement thermal contact pad with the gown assembly.

25-99. (canceled)
